(19) United States
(12) Patent Application Publication
(10) Pub. No.: US 2019/0083398 A1
(43) Pub. Date: Mar. 21, 2019

(54) RGD PEPTIDE AND PENETRATING PEPTIDE R8 CO-MODIFIED ERGOSTEROL AND CISPLATIN ACTIVE DRUG-LOADING LIPOSOME

(71) Applicant: ZheJiang Chinese Medical University, HangZhou, ZheJiang (CN)

(72) Inventors: Shengwu HUANG, HangZhou, ZheJiang (CN); Ting HUANG, HangZhou, ZheJiang (CN); Meijia WU, HangZhou, ZheJiang (CN); Dandan ZHAO, HangZhou, ZheJiang (CN)

(21) Appl. No.: 16/065,285

(22) PCT Filed: Apr. 5, 2016
(86) PCT No.: PCT/CN2016/078429

§ 371 Date: Jun. 22, 2018

(30) Foreign Application Priority Data
Mar. 11, 2016 (CN) 201610141181.9

(51) Int. Cl. A61K 9/127; A61K 31/575; A61K 33/24; A61K 47/42

(52) U.S. Cl. CPC A61K 9/127; A61K 31/575; A61K 9/1278; A61K 47/42; A61K 33/24

(57) ABSTRACT

The presently disclosed subject matter is directed to an RGD peptide and penetrating peptide R8 co-modified ergosterol and cisplatin active drug-loading liposome that is prepared by means of the incubation of an ergosterol and cisplatin active drug-loading liposome, RGD cyclic peptide, and penetrating peptide R8 in a water bath. The ergosterol and cisplatin active drug-loading liposome is prepared from an ergosterol liposome and a cisplatin solution serving as the raw materials. The ergosterol liposome is prepared from 8 wt % to 15 wt % ergosterol and 85 wt % to 92 wt % liposomes, and the liposomes consist of lecithin and cholesterol.

### TECHNICAL FIELD

The invention relates to a medicine for treating lung cancer, and more specifically, to an RGD peptide and penetrating peptide R8 co-modified ergosterol and cisplatin active drug-loading liposome.

### BACKGROUND

### Description of Related Art

Antrodia camphorata mainly contains chemical compositions such as polysaccharides, triterpenoids, proteins, vitamins and trace elements, as well as superoxide dismutase (SOD), adenosine, nucleic acid, lectin, amino acids, sterols, lignin, and blood pressure stabilizing substances. Triterpenoids are considered to be one of the only sources of bitterness in antrodia camphorata, found in mycelia and fruit bodies. So far, nearly 30 triterpenoids have been discovered, being mainly of two parental structures of lanostane and ergosterone.

The earliest human consumption of antrodia camphorata was to relieve hangovers and treat liver diseases. In recent years, antrodia camphorata has also been widely studied so as to further confirm the anti-liver cancer and hepatoprotective effects of antrodia camphorata. Another hot spot of the study is the anti-tumor effects of antrodia camphorata. In addition to liver cancer, breast cancer, colon cancer and oral cancer are also included. Xu Taihao et al. conducted a general analysis on the related anthropogenic papers of Taiwanese antrodia (from 1992 to 2010) and found that the study on the biological activity of antrodia camphora can be divided into 24 categories (see Table 1). The top five most studied categories are: (1) anti-tumor, (2) liver protection, (3) anti-oxidation, (4) immune regulation, and (5) anti-inflammation, showing the main pharmacological activity of antrodia camphorata. In addition, antrodia camphorata also shows some pharmacological activity on cardiovascular and cerebrovascular diseases, hypoglycemic and hypolipidemic treatments. Although the prior art reported that antrodia camphorata can resist liver cancer, breast cancer, colon cancer and oral cavity cancer, it has not been found to have a good anti-lung cancer effect. In addition, although antrodia camphorata has been recognized as having an anti-cancer effect, it is not clear what specific compositions play a part in the targeted anti-cancer effect due to the complicated composition of antrodia camphorata, which greatly hinders the study of anti-cancer drugs.

Liposomes are lipid bilayer microvesicles which resemble biofilm structures. At present, the preparation methods mainly include a passive drug loading method and an active drug loading method, wherein the active drug loading method overcomes the initial burst release and leakage of encapsulated drugs because of its high encapsulation ratio and low leakage of amphiphilic drug liposomes, particularly having a clinical value.

Cisplatin (CDDP), a complex of heavy metal platinum, is a bifunctional alkylating agent, and its chemical name is cis-dichlorodiammine platinum (II). It is a yellow crystalline powder which is slightly soluble in water, insoluble in general organic solvents such as ethanol, and soluble in dimethylformamide First synthesized by M. Peyrone in 1845, it was approved by the FDA for the clinical treatment of cancer in 1978. The discovery of cisplatin has led to the development of metal complexes in the medical field and has a revolutionary significance for cancer treatment. The anti-cancer effects include: (1) cisplatin is an efficient broad-spectrum anti-tumor drug which can interact with target DNA to form a CDDP-DNA complex and is a non-specific cell cycle drug; the tumor inhibition ratio is 61% to 98%, especially for solid tumors and tumors which are not sensitive to general chemotherapy drugs; (2) cisplatin not only can kill tumor cells and inhibit cell repair, but also has a strong radiosensitivity; (3) cisplatin has synergistic effects with various anti-cancer drugs, and its toxicity spectrum is also different from these anti-cancer drugs without cross-resistance, so cisplatin is easily compatible with other anti-tumor drugs, which is not only beneficial to the clinical combination of drugs, but also can reverse the toxicity of combination chemotherapy. Therefore, cisplatin has had a prominent position in anti-cancer drugs for a long time.

However, the clinical cisplatin preparations used currently, such as cisplatin for injection in the Chinese Pharmacopoeia (2010 version) and the European Pharmacopoeia (2001 version), and cisplatin for injection and cisplatin injections in the British Pharmacopoeia (2000 version), are all general injections for cancerous tissues and cells without selectiveness. The drug has a bioavailability with great side effects. The main problems are as follows: (1) serious toxicity and side effects: cisplatin and its metabolites are mainly excreted from the kidneys, having a great nephrotoxicity, as well as gastrointestinal toxicity, ototoxicity and neurotoxicity which cannot be ignored; (2) low activity on some cancer cells, such as breast cancer and colon cancer; (3) tendency to produce drug resistance; (4) being only slightly soluble in water, unstable in nature, and decomposed by light: the aqueous solution will be hydrolyzed after being placed at room temperature and it will fail, and it will turn into a toxic anti-platinum without any anti-tumor effect.

In view of the fact that the incidence and death ratio of lung cancer are highest among all types of cancers in recent years, and the ratio is still rising, how to reduce the toxicity and side effects of cisplatin has become an urgent problem to be solved.

### SUMMARY

The objective of the present invention is to provide an RGD peptide and penetrating peptide R8 co-modified ergosterol and cisplatin active drug-loading liposome, which partially replaces cisplatin with ergosterol to exert efficacy, ensuring an anti-lung cancer effect while significantly reducing the toxicity and side effects of the drug so as to have little harm to the human body; meanwhile, it uses an RGD peptide and penetrating peptide R8 as the target, with good targeting and drug effects.

The technical solution adopted in the present invention to solve the technical problems is as follows:

an RGD peptide and penetrating peptide R8 co-modified ergosterol and cisplatin active drug-loading liposome, characterized in that it is prepared by means of the incubation of an ergosterol and cisplatin active drug-loading liposome, and RGD cyclic peptide and penetrating peptide R8 in a water bath. The ergosterol and cisplatin active drug-loading liposome is prepared from an ergosterol liposome and a cisplatin solution serving as raw materials, wherein the mass ratio of ergosterol and cisplatin is controlled at 1:1-4:1.

After a long-term study, the inventor unexpectedly discovered that ergosterol has a significant anti-lung cancer effect, with low cytotoxicity and little harm to the human body. A simple ergosterol cannot reach the lesion directly after entering the body. Therefore, the ergosterol is encapsulated in the liposome, so that it can reach the lesion to exert its effect, and the targeting of the liposome enables the ergosterol to exert a better anti-lung cancer effect. Although cisplatin has a good anti-cancer effect, it has extreme toxicity and side effects, which greatly limits the application of cisplatin. Ergosterol is a naturally occurring compound in plants, with little cytotoxicity. The present invention adopts a newly discovered ergosterol with a better anti-cancer effect and low toxicity to partially replace cisplatin, acting synergistically with cisplatin to ensure an anti-lung cancer effect, while significantly reducing the toxicity and side effects of the drug, causing little harm to the human body and having a targeting ability.

In order to better exert the effect of the drug, and make it more targeted and more accurately reachable to the lesions directly, the present invention further modifies the ergosterol and cisplatin active drug-loading liposome, and attaches an RGD peptide and penetrating peptide R8 target, which enables the drug to have an excellent targeting ability and exert a good effect.

Preferably, the ergosterol liposome is made of ergosterol (8-15 wt %) and liposome (85-92%), wherein the liposome is composed of lecithin and cholesterol, with a molar ratio of lecithin to cholesterol of 3:1-6:1. Preferably, the ergosterol liposome is made of ergosterol (10 wt %) and liposome (90%), wherein the liposome is composed of lecithin and cholesterol, with a molar ratio of lecithin to cholesterol of 5:1.

Preferably, the amounts of RGD cyclic peptide and penetrating peptide R8 are controlled to be in a molar ratio of RGD cyclic peptide:penetrating peptide R8:cholesterol of 0.07:0.07:1.

Preferably, the RGD cyclic peptide is specifically DSPE (distearoylphosphatidylethanolamine)-PEG3400-c; the penetrating peptide R8 is specifically DSPE-PEG1000-R8. The RGD cyclic peptide and penetrating peptide R8 are commercially available or self-made, and the manufacturer selling the products is Shanghai Qiangyao Biotech Co., Ltd.

Preferably, the RGD peptide and penetrating peptide R8 co-modified ergosterol and cisplatin active drug-loading liposome is prepared by means of incubation of an ergosterol and cisplatin active drug-loading liposome, RGD cyclic peptide and penetrating peptide R8 in a water bath at 55° C. for 1 h.

Preferably, the cisplatin solution has a concentration of 0.03-0.3 mg/mL.

Preferably, the cisplatin solution has a concentration of 0.15 mg/mL.

Preferably, a specific method of preparing the ergosterol and cisplatin active drug-loading liposome is as follows:

(1) preparation of ergosterol liposome: lecithin, cholesterol and ergosterol are weighed, dissolved in chloroform, rotavaporized into a thin film, dried in a vacuum, hydrated with an ammonium chloride solution as a hydration solution and ultrasonically released. A probe is ultrasonically treated in an ice bath, filtered and extruded under a high pressure to generate an ergosterol liposome;

(2) gradient formation of ammonium chloride: the ergosterol liposome is added to a dialysis bag with a molecular weight cut off of 8000-14000 Da. The dialysis bag is closed and dialyzed in distilled water for 2 h, and the distilled water is changed once to continue the dialysis for 2 h;

(3) drug loading: cisplatin is mixed with water to make a cisplatin solution, and the dialyzed ergosterol liposome is added to the cisplatin solution for incubation, wherein the amount of ergosterol liposome added satisfies the mass ratio of ergosterol to cisplatin being between 1:1 to 4:1, and the product is generated after the incubation at an incubation temperature of 40-60° C. and an incubation time of 5-40 min

When the cisplatin solution is encapsulated by hydration as a hydration solution, the encapsulation ratio is found to be less than 10%, indicating that the film dispersion method is unsuitable for encapsulating such a cisplatin. Cisplatin is a weakly basic drug, and the invention is prepared by an ammonium chloride gradient method, with an encapsulation ratio of up to more than 50%.

The main process of the ammonium chloride gradient method of preparing an active drug-loading liposome is as follows: preparation of an ergosterol liposome and gradient formation of ammonium chloride, and drug loading, wherein the gradient formation of ammonium chloride is important. The basic principle is that a certain concentration of ammonium chloride is encapsulated in the internal aqueous phase of the liposome, and the external aqueous phase of ammonium chloride is removed by dialysis. Due to the difference of internal and external concentrations, the diffusion coefficient of ammonia molecules is much higher than that of ammonium chloride. With the diffusion of ammonia molecules, the liposomes gradually protonate internally, so that a pH gradient is formed indirectly from the ammonium chloride gradient. At this gradient, cisplatin exists in a molecular state in the external aqueous phase, having a strong penetrating capacity, and exists in an ionized state in the internal aqueous phase, being difficult to diffuse, thus resulting in a stable encapsulation state.

Preferably, the parameters of the ultrasonic treatment of the probe in Step (1) are as follows: ultrasonic time: 20 min, ultrasonic: 2 s, stop: 1 s, ultrasonic power: 900W and pressure for the high-pressure extrusion: 400-500 psi.

Preferably, the amount of the ergosterol liposome added in Step (3) is calculated to satisfy the mass ratio of the ergosterol to cisplatin being 2.5:1, the incubation temperature is 50° C. and the incubation time is 10 min

Preferably, the concentration of the ammonium chloride solution in Step (1) is 0.1-1.5 mmol·L⁻¹.

The beneficial effects of the present invention are as follows: ergosterol is used to partially replace cisplatin to exert efficacy, ensuring an anti-lung cancer effect while significantly reducing the toxicity and side effects of the drug so as to have little harm to the human body; meanwhile, it uses an RGD peptide and penetrating peptide R8 as the target, with good targeting and drug effects.

### BRIEF DESCRIPTION OF THE DRAWINGS

FIGS. 1A-1C are transmission electron microscopy (TEM) images of a blank liposome, an ergosterol liposome, and an ergosterol and cisplatin liposome, respectively, at 50,000×.

FIG. 2 is a line graph showing the cumulative release of the ergosterol and cisplatin active drug-loading liposomes.

FIG. 3 is a bar graph showing the results of 24 h MTT assay of the drug substances and liposomes, wherein ※※P<0.01 when compared with the ergosterol cisplatin liposome group with the same dilution factor.

FIGS. 4a-4d are TEM images of unmodified liposomes, RGD modified liposomes, R8 modified liposomes, and RDG +R8 modified liposomes, respectively.

FIGS. 5a and 5b are FV 1000 laser confocal microscopy images of the penetration of tumor balls by co-modified liposomes at pH 6 and pH 7.4, respectively.

FIG. 6 is a bar graph showing the uptake inhibitory ratio (%) of different cellular uptake inhibitors, wherein **P<0.01, (n=3) when compared to the control group.

FIG. 7 is a bar graph showing inhibition ration (%) versus dilution factor for the results of 2 h MTT assays of each targeted liposome.

FIG. 8 is a bar graph showing inhibition ratio (%) versus dilution factor for the results of 24 h MTT assay of each targeted liposome.

### DETAILED DESCRIPTION

The technical solution of the present invention is further detailed through the embodiments in combination with the drawings as below.

In the present invention, if not specified, the raw materials and the equipment used may be commercially available or commonly used in the field. The methods in the following embodiments are all conventional methods in the art unless otherwise specified. Embodiments:

An RGD peptide and penetrating peptide R8 co-modified ergosterol and cisplatin active drug-loading liposome is prepared by means of the incubation of an ergosterol and cisplatin active drug-loading liposome, RGD cyclic peptide (DSPE-PEG3400-c, commercially available) and penetrating peptide R8 (DSPE-PEG1000-R8, commercially available) in a water bath at 55° C. for 1 h. The ergosterol and cisplatin active drug-loading liposome is prepared from an ergosterol liposome and a cisplatin solution serving as raw materials, wherein the mass ratio of the ergosterol and cisplatin is controlled at 1:1-4:1. The ergosterol liposome is made of ergosterol (8-15 wt %) and liposome (85-92%), wherein the liposome is composed of lecithin and cholesterol, with a molar ratio of lecithin to cholesterol of 3:1-6:1. The amounts of RGD cyclic peptide and penetrating peptide R8 are controlled to be in a molar ratio of RGD cyclic peptide:penetrating peptide R8:cholesterol of 0.07:0.07:1. The cisplatin solution has a concentration of 0.03-0.3 mg/mL.

The method of preparing an ergosterol and cisplatin active drug-loading liposome comprises the steps as follows:

(1) preparation of ergosterol liposome: lecithin, cholesterol and ergosterol are weighed, dissolved in chloroform, rotavaporized into a thin film, dried in a vacuum, hydrated with an ammonium chloride solution (the concentration of 0.1-1.5 mmol·L-1) as a hydration solution and ultrasonically released. A probe is ultrasonically treated in an ice bath, filtered and extruded under a high pressure to generate an ergosterol liposome. The raw material compositions of the ergosterol liposome are matched at a sum of 100% as follows: ergosterol: 5-20 wt % and the molar ratio of the remaining lecithin to cholesterol is 1:1-7:1. The parameters of the ultrasonic treatment of the probe are as follows: ultrasonic time: 20 min, ultrasonic: 2 s, stop: 1 s, ultrasonic power: 900W and pressure for the high-pressure extrusion: 400-500 psi;

(2) gradient formation of ammonium chloride: the ergosterol liposome is added to a dialysis bag with a molecular weight cut off of 8000-14000 Da. The dialysis bag is closed and dialyzed in distilled water for 2 h. The distilled water is changed once to continue the dialysis for 2 h;

(3) drug loading: cisplatin is mixed with water to make a cisplatin solution (the cisplatin solution has a concentration of 0.03-0.3 mg/mL), and the dialyzed ergosterol liposome is added to the cisplatin solution for incubation, wherein the amount of ergosterol liposome added satisfies the mass ratio of ergosterol:cisplatin being between 1:1 to 4:1, and the product is generated after the incubation at an incubation temperature of 40-60° C. and an incubation time of 5-40 min

1. Best Preparation Process of Ergosterol Liposome

1.1 Examination of a Single Factor

1.1.1 Examination of the Molar Ratio of Lecithin and Cholesterol

The test examines the ratio of lecithin to cholesterol (molar ratio) when ergosterol is loaded at 5%. The ratio of lecithin to cholesterol is set at 1:1, 3:1, 5:1 and 7:1, and the encapsulation ratio is 71.59%, 89.15%, 92.58% and 96.62%. When the ratio of lecithin to cholesterol is 1:1, the rigidity of the liposome increases due to the higher ratio of cholesterol, and when the ratio is 7:1, sedimentation occurs easily after being placed.

1.1.2 Examination of Ultrasonic Probe Time

After the liposomes are prepared by means of a thin-film dispersion method (lecithin, cholesterol and ergosterol are weighed, dissolved in chloroform, rotavaporized into a thin film, dried in a vacuum, hydrated and ultrasonically released), the whole liposomes with a large particle size are turned into liposomes with a small particle size via an ultrasonic probe. The test examines the effect of different ultrasonic probe times on the encapsulation ratio of the ergosterol liposome. The ultrasonic time of the probe is set to 10 min, 20 min, 30 min and 40 min, and the encapsulation ratios are 79.45%, 91.73%, 95.86% and 95.94%, respectively. With the increase of time, the encapsulation ratio increases, and the encapsulation ratio no longer changes at 30 min In addition, the prolonged ultrasonic time may easily lead to a liposome rupture.

1.1.3 Examination of the Drug Loading of Ergosterol

The test examines the effect of different drug loadings of ergosterol on the encapsulation ratio. The ergosterol drug loadings are 5%, 10%, 15% and 20%, and the encapsulation ratios are 90.76%, 85.81%, 69.79% and 73.09%, respectively.

1.2 Response Surface Test

1.2.1 Star-Point Design

The results of a single factor test show that the ratio of lecithin to cholesterol, ultrasonic probe time, and drug loading have a significant effect on the encapsulation ratio of ergosterol. According to the principle of a star-point design, the ratio of lecithin to cholesterol, ultrasonic probe time and drug loading are selected as independent variables, wherein each independent variable is identified as three levels represented by the Codes—1, 0, and 1, respectively, among a total of 15 test points (3 central points). Each test is performed for three times in parallel. The encapsulation ratio of ergosterol liposome is used as an evaluation index, and the star-point design (Table 1) is used to optimize the preparation process conditions.

TABLE 1
Star-point test arrangement for preparation
of ergosterol liposomes (n = 3)

| | X₁-Ratio | X₂-Ultrasonic | | Encapsulation |
|---|---|---|---|---|
| | of lecithin to | probe | X3 drug | ratio of |
| No. | cholesterol | time/min | loading/% | ergosterol/% |
| 1 | 3.00 | 5.00 | 40.00 | 88.77 |
| 2 | 5.00 | 10.00 | 20.00 | 72.77 |
| 3 | 1.00 | 10.00 | 20.00 | 52.35 |
| 4 | 5.00 | 15.00 | 30.00 | 73.94 |
| 5 | 1.00 | 15.00 | 30.00 | 50.67 |
| 6 | 5.00 | 5.00 | 30.00 | 92.90 |
| 7 | 3.00 | 10.00 | 30.00 | 88.35 |
| 8 | 1.00 | 10.00 | 40.00 | 46.69 |
| 9 | 3.00 | 5.00 | 20.00 | 85.17 |
| 10 | 1.00 | 5.00 | 30.00 | 39.13 |
| 11 | 3.00 | 10.00 | 30.00 | 90.06 |
| 12 | 3.00 | 10.00 | 30.00 | 92.78 |
| 13 | 3.00 | 15.00 | 40.00 | 88.22 |
| 14 | 3.00 | 15.00 | 20.00 | 72.50 |
| 15 | 5.00 | 10.00 | 40.00 | 92.22 |

1.2.2 Establishment of Model and Analysis of Variance

The Design-Expert.V 8.0.6.1 software is used to perform a quadratic multivariate regression fitting on the data in the table, thus generating a regression equation of the independent variable and the dependent variable: Y=90.40+17.87X₁−2.58X₂+4.14X₃−7.62X₁X₂+6.28X₁X₃+3.03X₂X₃−21.95X₁²−4.29X₂²−2.44X₃². The regression model is tested for significance. The results in Table 2 show that X₁ and X₁² have a very significant linear effect on the response values, and X₂, X₃ and X₂² have a significant curve effect on the response values as well as the interaction items of X₁X₂ and X₁X₃. In the model, F=82.84 and P<0.0001 indicate that the quadratic multivariate regression model is extremely significant, and the regression equation correlation coefficient (r) of 0.9967 indicates that the model can explain 99.67% of the response value changes, with a good fitting.

TABLE 2
Analysis of variance of regression model

| Source of variance | SS | f | MS | F | P |
|---|---|---|---|---|---|
| Model | 4977.80 | 9 | 553.09 | 82.84 | <0.0001 |
| X₁-Ratio of lecithin to | 2555.77 | 1 | 2555.77 | 382.79 | <0.0001 |
| cholesterol | | | | | |
| X₂-Drug loading | 53.25 | 1 | 53.25 | 7.98 | 0.0369 |
| X₃-Ultrasonic probe | 137.03 | 1 | 137.03 | 20.52 | 0.0062 |
| time | | | | | |
| X₁X₂ | 232.56 | 1 | 232.56 | 34.83 | 0.0020 |
| X₁X₃ | 157.63 | 1 | 157.63 | 23.61 | 0.0046 |
| X₂X₃ | 36.72 | 1 | 36.72 | 5.50 | 0.0659 |
| X₁² | 1778.49 | 1 | 1778.49 | 266.37 | <0.0001 |
| X₂² | 67.94 | 1 | 67.94 | 10.18 | 0.0243 |
| X₃² | 22.02 | 1 | 22.02 | 3.30 | 0.1291 |
| Error | 33.38 | 5 | 6.68 | | |
| Lack of fit | 23.40 | 3 | 7.80 | 1.56 | 0.4131 |
| Pure error | 9.98 | 2 | 4.99 | | |
| Total dispersion | 5011.18 | 14 | | | |

1.2.3 Analysis of the Response Surface

According to a regression equation, when the code value of 1 factor is maintained at 0, a three-dimensional response surface map of the relationship between the other 2 factors and the encapsulation ratio of ergosterol is plotted using the Design-Expert.V 8.0.6.1 software. The response surface is a three-dimensional space curve formed by the response value to the pairwise interaction factors. The steeper the effect surface curve is, the more obvious the influence of each variable on the response value is. The maximum point of the regression model is taken, and the corresponding measurement value of lecithin to cholesterol is 5:1, the ultrasonic probe time is 20 min and the ergosterol drug loading is 10%.

1.2.4 Verification Test

To verify the applicability of the model equation, lecithin (98 mg), cholesterol (10 mg) and ergosterol (12 mg), in total 3 copies, are accurately weighed for a verification test performed according to the following best preparation process conditions. The results show that the average encapsulation ratios of the three batches of liposomes are 90.49%, with an RSD of 2.64% and a variance of 0.10% from the predicted value of 90.40%, indicating that the established mathematical model has good predictability, and the preferable process conditions have good repeatability.

The best preparation process of the ergosterol liposome is as follows: the molar ratio of lecithin to cholesterol is 5:1, the drug loading of ergosterol is 10% and the ultrasonic probe is 20 min

The specific process conditions are as follows: lecithin (98 mg), cholesterol (10 mg) and ergosterol (12 mg) are fully dissolved in 10 mL of chloroform, rotavaporized on a rotary evaporator in a water bath at 40° C., dried in a vacuum for 2 h, added with 10 mL of ammonium chloride solution (0.5 mmol·L^{31 1}) and hydrated on a horizontal shaker at room temperature for 30 min at a rotation speed of 140 rpm·mL⁻¹. After hydration, the films are removed by ultrasonic probe and the liposomes are placed in an ice bath (ultrasonic probe: 20 min, ultrasonic time: 2 s, stop: 1 s, ultrasonic power: 900W). The liposomes are filtered through 0.8 μm, 0.45 μm and 0.22 μm microporous films and finally extruded with a 0.1 μm polycarbonate film (pressure: 400-500 psi).

2. Examination of the Best Preparation Process of Ergosterol and Cisplatin Active Drug-Loading Liposome

2.1 Single Factor Test

2.1.1 Examination of the Mass Ratio of Ergosterol/Cisplatin

The test examines the mass ratio of ergosterol to cisplatin, equivalent to the drug loading of cisplatin in liposomes. After the ergosterol liposome is prepared in the best process according to 1.2.4, the ergosterol liposome is added to a dialysis bag with a molecular weight cut off of 8000-14000 Da. The dialysis bag is closed and dialyzed in distilled water for 2 h, and the dialysate (distilled water) is changed once to continue the dialysis for 2 h. The ergosterol liposome is mixed with different concentrations of cisplatin solution for incubation, with the mass ratios of ergosterol liposome to cisplatin set to 0.313:1, 0.625:1, 1.25:1, 2.5:1 and 5:1. The results show that when the ratio of ergosterol liposome to cisplatin is 2.5:1, the encapsulation ratio of cisplatin is the highest, reaching 35.33%.

2.1.2 Examination of Incubation Time

In the test, the encapsulation ratios of cisplatin solution at 5, 10, 20 and 40 min are examined The results show that when the incubation time is 20 min, the encapsulation ratio of cisplatin is the highest, reaching 31.07%.

2.1.3 Examination of Incubation Temperature

The test examines the influence of temperatures at 40, 50, 60 and 80° C. on the encapsulation ratio of cisplatin. The results show that the encapsulation ratios at 50° C. and 80° C. are the highest, but an excessively high temperature will cause the oxidation of lecithin which produces hemolysis.

2.2 Orthogonal Test

On the basis of the examination of a single factor test, the ergosterol and cisplatin active drug-loading liposome is optimized using a three-factor and three-level orthogonal design, and an L₉(3⁴) orthogonal table is used to arrange the test. Three factors of incubation time, incubation temperature, and cisplatin concentration are selected as the examination factors to determine the best preparation process of an ergosterol and cisplatin active drug-loading liposome. The orthogonal design factor level table, the orthogonal design test plan, and the test results are shown in Table 3 and Table 4.

TABLE 3
L₉(3⁴) orthogonal table

| | A-Incubation | B-Incubation | C-Cisplatin |
|---|---|---|---|
| | time | temperature | concentration |
| | (min) | (° C.) | (mg mL⁻¹) |
| 1 | 10 | 30 | 0.150 |
| 2 | 20 | 50 | 0.075 |
| 3 | 40 | 70 | 0.037 |

TABLE 4
L₉(3⁴) test plan table

| | A- | B- | | | Encapsu- |
|---|---|---|---|---|---|
| | Incubation | Incubation | C-Cisplatin | D- | lation |
| Test | time | temperature | concentration | Error | ratio |
| No. | (min) | (° C.) | (mg mL⁻¹) | term | (%) |
| 1 | 1(10) | 1(30) | 1(0.150) | 1 | 26.93 |
| 2 | 1(10) | 2(50) | 2(0.075) | 2 | 17.25 |
| 3 | 1(10) | 3(70) | 3(0.037) | 3 | 17.21 |
| 4 | 2(20) | 1(30) | 2(0.075) | 3 | 10.55 |
| 5 | 2(20) | 2(50) | 3(0.037) | 1 | 13.67 |
| 6 | 2(20) | 3(70) | 1(0.150) | 2 | 18.57 |
| 7 | 3(40) | 1(30) | 3(0.037) | 2 | 7.89 |
| 8 | 3(40) | 2(50) | 1(0.150) | 3 | 19.26 |
| 9 | 3(40) | 3(70) | 2(0.075) | 1 | 19.33 |
| Average | 20.463 | 15.123 | 21.587 | 19.977 | |
| 1 | | | | | |
| Average | 14.263 | 16.727 | 15.710 | 14.570 | |
| 2 | | | | | |
| Average | 15.493 | 18.370 | 12.923 | 15.673 | |
| 3 | | | | | |
| Range | 6.200 | 3.247 | 8.664 | 5.407 | |

A range analysis method is performed using single-indicator orthogonal design test results. The results show that the degree of influence of each factor on the encapsulation ratio of cisplatin is C>A>B. The best combination is the ergosterol and cisplatin active drug-loading liposome and the best process is A₁B₃C₁, that is, the concentration of cisplatin is 0.150 mg·mL⁻¹, the incubation temperature is 70° C. and the incubation time is 10 min Considering that 70° C. is above the phase transition temperature of soy lecithin, and the factor of incubation temperature has little effect on the encapsulation ratio, the best process is adjusted to: the concentration of cisplatin is 0.150 mg·mL⁻¹, the incubation temperature is 50° C. and the incubation time is 10 min; the best process before and after adjustment is verified.

2.3 Verification of Orthogonal Test

Three batches of ergosterol and cisplatin active drug-loading liposomes are prepared according to the best process of orthogonal test and the best process after adjustment. The encapsulation ratio, the average particle size, and the Zeta potential of cisplatin are measured by ultrafiltration. The results of the average encapsulation ratio of 3 batches of liposomes are 49.04% and 52.24%, respectively. The results in Table 5 and Table 6 show that there is no significant difference in the encapsulation ratio, the average particle size and the Zeta potential between the best process and the best process after adjustment; therefore, the subsequent test uses the best process after adjustment, that is, the ergosterol and cisplatin active drug-loading liposome is prepared when the concentration of cisplatin is 0.150 mg·mL⁻¹, the incubation temperature is 50° C. and the incubation time is 10 min

TABLE 5
Best process verification results

| | | Average | Average | | |
|---|---|---|---|---|---|
| | Encapsulation | encapsulation | particle | | Zeta |
| Batch | ratio | ratio | size | | potential |
| No. | (%) | (%) | (nm) | PDI | (mV) |
| 1 | 54.94 | 49.04 | 151.9 | 0.159 | −30.2 |
| 2 | 44.89 | | 150.0 | 0.138 | −31.9 |
| 3 | 47.30 | | 152.8 | 0.170 | −31.8 |

TABLE 6
Verification results of best process after adjustment

| | | Average | Average | | |
|---|---|---|---|---|---|
| | Encapsulation | encapsulation | particle | | Zeta |
| Batch | ratio | ratio | size | | potential |
| No. | (%) | (%) | (nm) | PDI | (mV) |
| 1 | 51.81 | 52.24 | 152.7 | 0.107 | −36.9 |
| 2 | 56.13 | | 151.3 | 0.148 | −34.1 |
| 3 | 48.79 | | 154.1 | 0.119 | −35.6 |

3. Quality Evaluation of Ergosterol and Cisplatin Active Drug-Loading Liposome

3.1 Morphological Observation

3.1.1 Appearance

The ergosterol and cisplatin active drug-loading liposome solution is milky white with a uniform color.

3.1.2 Microscopic Morphology

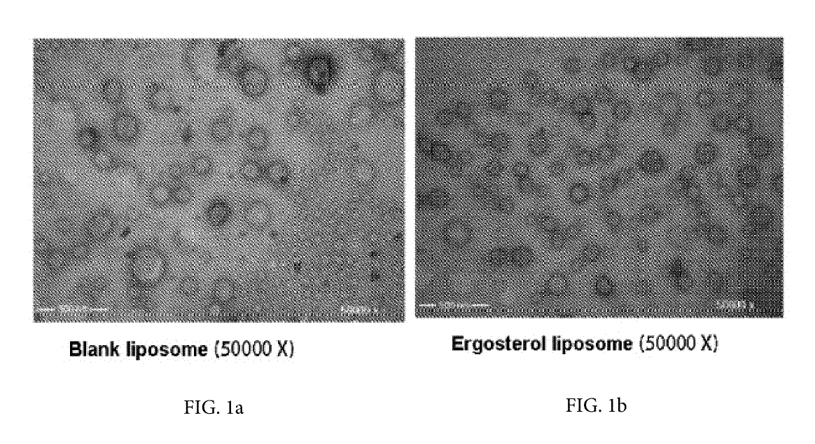

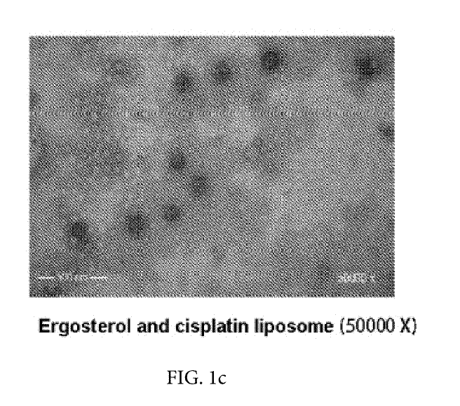

A sample is prepared by means of negative staining. At room temperature, an ergosterol and cisplatin active drug-loading liposome is taken, diluted with distilled water to be slightly cloudy and dripped onto a dedicated 230-mesh copper grid. The excess liposomes are dried with a filter paper, standing for lmin The ergosterol and cisplatin active drug-loading liposome is negatively stained with 1% phosphotungstic acid, standing for 40s, so that the particles are deposited on the copper mesh. The excess dye liquid on the edges of the copper mesh is removed with a filter paper, spontaneously evaporated, and then observed with an electron microscope and photographed. The results in FIG. 1 show that each liposome has a round morphology and a uniform distribution of particle size.

3.1.3 Encapsulation Ratio and Drug Loading

Among three batches of ergosterol and cisplatin active drug-loading liposomes, the average encapsulation ratio of ergosterol is 90.49% and the drug loading is 0.1401 mg·mL⁻¹. The average encapsulation ratio of cisplatin is 52.24% and the drug loading is 0.1382 mg·mL⁻¹.

3.1.4 Particle Size and its Distribution

At room temperature, an ergosterol and cisplatin active drug-loading liposome is taken and diluted 20 times, injected into a sample cell, and the average particle size and its distribution are measured with a laser particle size analyzer. The results show that the average particle size of the blank liposome is 145.8 nm, and the polydispersity coefficient PDI is 0.168, less than 0.3. The average particle size of the ergosterol liposome is 131.4 nm, and the PDI is 0.152, less than 0.3. The average particle size of the ergosterol and cisplatin active drug-loading liposome is 112.5 and the PDI is 0.208, less than 0.3. It is clear that the particle size distribution of the blank liposome and the ergosterol liposome is more concentrated.

3.1.5 Zeta Potential Measurement

At room temperature, an ergosterol and cisplatin active drug-loading liposome is taken and diluted 20 times, and the Zeta potential is measured by a Zeta potential analyzer. The results show that the zeta potential of the blank liposome is −18.6 mV, the zeta potential of the ergosterol liposome is −23.4 mV and the zeta potential of the ergosterol and cisplatin active drug-loading liposome is −5.42 mV, with the liposomes negatively charged.

3.1.6 pH Measurement

At room temperature, an ergosterol and cisplatin active drug-loading liposome is taken and diluted, and pH is measured with an acidometer. The average pH of three batches of samples are measured to be 6.64.

3.1.7 Measurement of the Peroxide Value (POV) of Ergosterol and Cisplatin Active Drug-Loading Liposome

Phospholipid molecules contain unsaturated fatty acid chains which are chemically unstable and easily oxidatively hydrolyzed to reduce the film fluidity, accelerate drug leakage, and produce peroxide products, such as malondialdehyde and fatty acids, which are toxic to humans. Malondialdehyde (MDA) reacts with thiobarbituric acid under acidic conditions, and the resulting red product (TBA-pigment) absorbs at 535 nm. The content of malondialdehyde can be obtained by measuring the absorbency so that the degree of oxidation of the phospholipid can be examined In the test, a malondialdehyde assay is used to examine the degree of liposome oxidation.

The liposome (1 mL) is accurately pipetted, placed in a 10 mL centrifuge tube, added with a TTH test solution (5 mL) (trichloroacetic acid (30 g) and 2-thiobarbituric acid (0.75 g) added with 0.25 mol·L⁻¹ of hydrochloric acid (200 mL), warmed to dissolve and filtered after cooling), uniformly mixed, heated in a water bath at 100° C. for 30 min, cooled to room temperature, added with 4.0 mL of a TTH test solution, uniformly mixed and centrifuged at 4000 rpm·min⁻¹ for 10 min The supernatant is pipetted, and the TTH test solution is used as a blank control. The absorbency is measured at a wavelength of 535 nm and recorded as a peroxide value. Three batches of samples are measured in parallel with an average peroxide value of 0.1095 (Table 7).

TABLE 7
Results of peroxide value (POV) of liposomes

| Batch No. | POV | Average POV |
|---|---|---|
| 1 | 0.1059 | 0.1095 |
| 2 | 0.1108 | |
| 3 | 0.1117 | |

3.1.8 Release Test

Since ergosterol has a strong lipophilicity and a very low solubility in the release medium, only the release of the water-soluble drug of cisplatin is examined in the test. An ergosterol and cisplatin active drug-loading liposome (1 mL) and a cisplatin drug substance solution (1 mL) are accurately pipetted, respectively, and placed in a dialysis bag, with both ends clamped with a dialysis clip. Since cisplatin is not stable in a sodium-free or low-sodium solution, it is easily hydrolyzed into an anti-platinum without an anti-cancer composition; therefore, the release medium is selected as a 100-time 0.9% NaCl solution and placed in a constant-temperature water bath shaker at a shaking speed of 100 rpm·min⁻¹, with a release temperature of 37° C.; a dialysate (1 mL) is pipetted at 0.5, 1, 2, 3, 4, 6, 8, 10, 12 and 24 h, respectively, and supplemented with a 0.9% NaCl fresh dialysis medium (1 mL) at 37° C. The samples at each sampling point are filtered through a 0.45 μm microporous film and injected for analysis. The area of the peak is measured by HPLC injection, and the drug release concentrations of cisplatin at each sampling point are calculated by substituting a linear regression equation, recorded as c₁. The total dose of cisplatin is recorded as M₀.

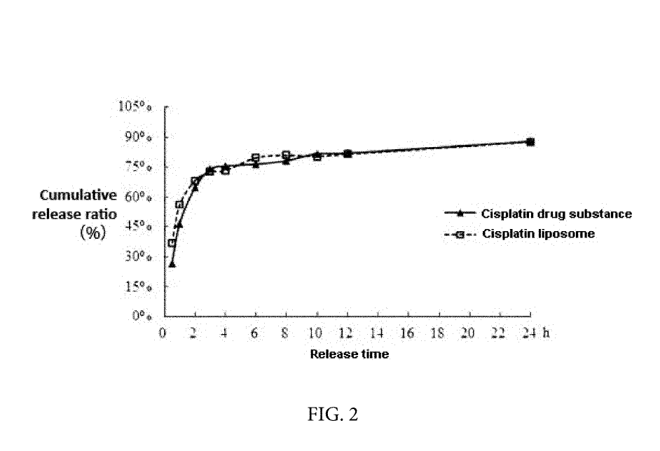

The calculation is performed according to the following formula: the

$$cumulative release ratio = ( {c}_{1} \times {V}_{0} + {\sum }_{n = 1}^{i - 1} cV ) / {M}_{0} \times 100 \% ;$$

wherein, c₁ is the concentration of cisplatin released at each sample point, V₀ is the volume of the medium released, V is the sampling volume, and M₀ is the total amount of cisplatin contained in the ergosterol and cisplatin active drug-loading liposome. The requirements for the burst effect of liposomes in the “General Rules of Preparation in the Appendix of the Chinese Pharmacopoeia”: if the initial release at 0.5 h is≤40%, the ergosterol and cisplatin active drug-loading liposome meets the requirements, and if the cumulative release ratio at 24 h is higher than 80%, the liposome meets the requirements (FIG. 2).

4. Cell Uptake Test of Ergosterol and Cisplatin Active Drug-Loading Lipsome

The prepared FITC-labeled co-modified liposome 1640 medium with a pH of 7.4 is mixed quantificationally, so that the final dilution factors are 64, 96 and 128. The A549 cells (commercially available, the Cell Bank of the Institute of Life Sciences of the Chinese Academy of Sciences) are inoculated in a 6-well plate. When the cells are fused to 80%, the mixture of the ergosterol and cisplatin active drug-loading liposome and the culture medium is pipetted following the former culture medium. The culture medium is pipetted at 37° C. in a 5% CO₂ incubator after incubation for 2 h, washed 3 times with PBS, trypsinized to collect cells and washed 3 times with PBS. The supernatant is removed by centrifugation and finally added with 0.5 mL of PBS to re-suspend the cells. The uptake intensity of the co-modified liposomes at different concentrations are measured by a BD flow cytometry. As shown in Table 8, the results show that given an ergosterol and cisplatin active drug-loading liposome, the cell uptake ratio decreases with the increase in the dilution factors.

TABLE 8
Cell uptake of ergosterol and cisplatin
active drug-loading liposome

| | | Uptake ratio |
|---|---|---|
| Groups | No. | (%) |
| Negative | 1 | 0.1% |
| control | 2 | 0.0% |
| | 3 | 0.1% |
| Diluted | 1 | 62.9% |
| 64 times | 2 | 74.5% |
| | 3 | 60.0% |
| Diluted | 1 | 46.9% |
| 96 times | 2 | 51.2% |
| | 3 | 49.9% |
| Diluted | 1 | 34.7% |
| 128 times | 2 | 35.1% |
| | 3 | 38.0% |

5. In Vitro Cell Proliferation Inhibition Test of Ergosterol and Cisplatin Active Drug-Loading Liposome

To verify that the ergosterol and cisplatin drug substances can be prepared into liposome preparations to enhance their anti-tumor activity, the test uses the A549 lung cancer cells cultured in vitro, stimulated with ergosterol, cisplatin, ergosterol and cisplatin drug substances, and ergosterol and cisplatin active drug-loading liposome preparations. The cell proliferation inhibition ratios are measured after administration at different concentrations, and the C50 values of the half-inhibition ratios of the liposome preparations on A549 cells are calculated.

The A549 cells in a logarithmic growth phase are taken, with the number adjusted to 1×10⁵·mL⁻¹ after a trypsin digestion. 100 μL of the A549 cells are added in per well and inoculated in a 96-well culture plate, incubated at 37° C. in a 5% CO₂ incubator, and added with drugs for treatment when the cells are fused to 80%. The ergosterol and cisplatin active drug-loading liposome with different dilution factors, as well as the corresponding ergosterol, cisplatin, and ergosterol and cisplatin drug substance solutions, are added, and the normal control group is also set. Five complex holes are set for each concentration, and the MTT assay is performed 24 h after dosing. An MTT (5 mg·mL⁻¹) solution (20 μL) is added to each well and incubated at 37° C. in a 5% CO₂ incubator for 4 h. The absorbency (OD) value in each well is measured at 492 nm with a microplate reader. The test is measured 3 times in parallel.

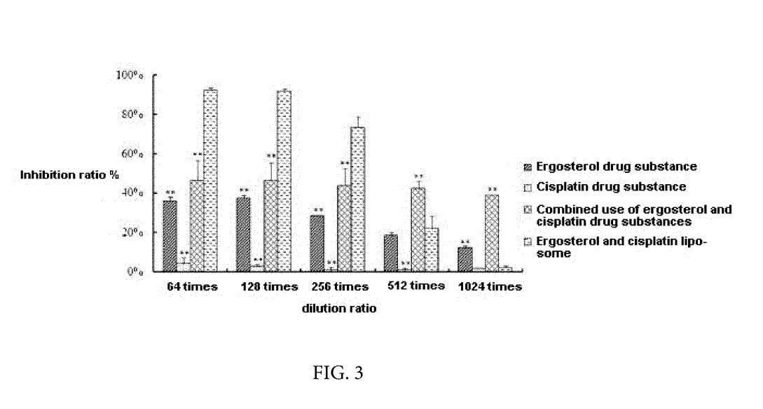

FIG. 3 shows that after the drug acts for 24 h, when the dilution factors are 64, 128 and 256 times, compared with the other three groups with the same dilution factor, the inhibition ratios are significantly higher in the ergosterol and cisplatin active drug-loading liposome group, with an extremely significant difference, and ※※P<0.01 (n=3). The IC₅₀ value of the administration of the ergosterol and cisplatin liposome is 2.178+0.544 μg·mL⁻¹. It shows that the preparation of ergosterol and cisplatin into liposome preparations is able to significantly increase its anti-lung cancer effect in vitro.

6. Preparation of RGD Peptide and Penetrating Peptide R8 Co-Modified Ergosterol and Cisplatin Active Drug-Loading Liposome

The preparation of an ergosterol and cisplatin active drug-loading liposome, according to the best preparation process of ergosterol and cisplatin active drug-loading liposome, uses a post-insertion method to prepare a simple penetrating peptide R8 modified liposome (R8-Lip) by means of incubation in a water bath at 55° C. for 1 h, with the molar ratio of penetrating peptide R8 (DSPE-PEG1000-R8, synthesized by Shanghai Shengyao Biotechnology Co., Ltd.):cholesterol of 0.07:1; to prepare a simple RGD modified liposome (RGD-Lip) by means of incubation in a water bath at 55° C. for 1 h, with the molar ratio of RGD cyclic peptide (DSPE-PEG3400-c, synthesized by Shanghai Qiangyao Biotechnology Co., Ltd.):cholesterol of 0.07:1; and to prepare a co-modified liposome (RGD with R8-Lip) by means of incubation at 55° C. for 1 h, with the molar ratio of RGD cyclic peptide (DSPE-PEG3400-c, synthesized by Shanghai Qiangyao Biotechnology Co., Ltd.):penetrating peptide R8 (DSPE-PEG1000-R8, synthesized by Shanghai Qiangyao Biotechnology Co., Ltd.):cholesterol of 0.07:0.07:1. For an FITC-labeled liposome, an appropriate amount of FITC methanol solution is added to the lipid material for rotary evaporation to form a thin film. The influence of the concentrations of different fluorescein isothiocyanates (FITC) on the cell uptake ratios is measured by a fluorescence spectrophotometer, with the results shown in Table 9. The final mass concentration of FITC in the liposome is measured to be 25 μg·mL⁻¹.

TABLE 9
Influence of FITC concentrations of different
fluorescent substances on cell uptake ratio

| | FITC final | | |
|---|---|---|---|
| | concentration | Uptake ratio | |
| No. | (μg · mL⁻¹) | (%) (x ± s) | Significant |
| 1 | 50 | 44.70% ± 0.92% | ** |
| 2 | 25 | 55.33% ± 0.17% | ** |
| 3 | 12.5 | 22.54% ± 0.09% | ** |
| 4 | 6.25 | 5.31% ± 0.05% | ** |
| 5 | 3.125 | 0.34% ± 0.08% | ** |

** P < 0.01, there are a significant differences between the groups.

7. Quality Evaluation of RGD Peptide and Penetrating Peptide R8 Co-Modified Ergosterol and Cisplatin Active Drug-Liposome

7.1 Microscopic Morphology

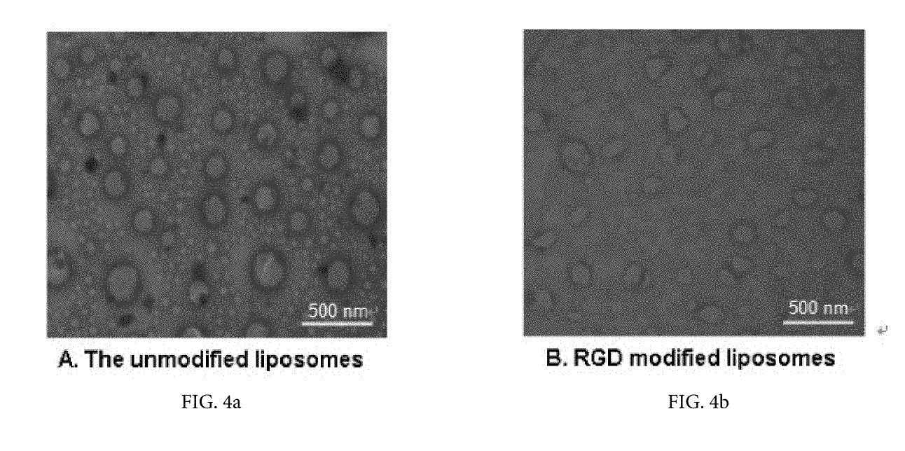

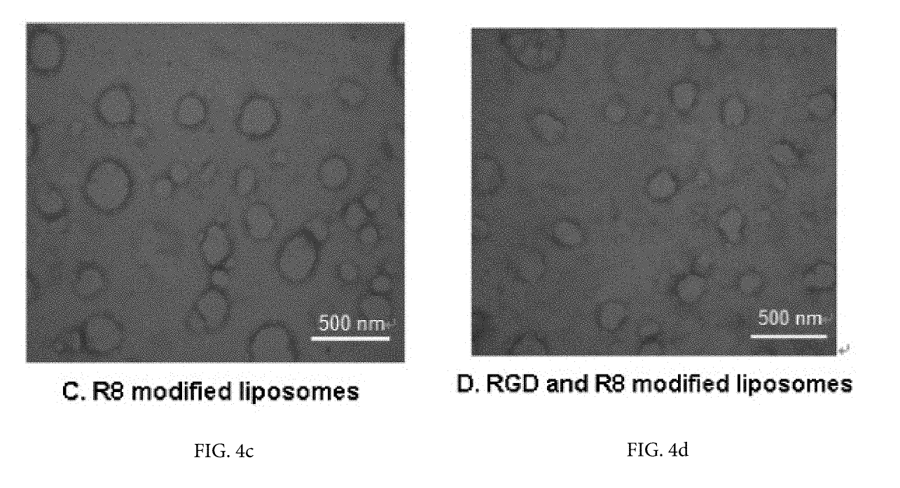

A sample is prepared by means of negative staining At room temperature, an ergosterol and cisplatin active drug-loading liposome is taken, diluted with distilled water to be slightly cloudy and dripped onto a dedicated 230-mesh copper grid. The excess liposomes are dried with a filter paper, standing for 1 min. The ergosterol and cisplatin active drug-loading liposome is negatively stained with 1% phosphotungstic acid, standing for 40 s, so that the particles are deposited on the copper mesh. The excess dye liquid on the edges of the copper mesh is removed with a filter paper, spontaneously evaporated, and then observed with an electron microscope and photographed (FIG. 4). The results show that each liposome has a round morphology with a bilayer structure and a uniform distribution of particle size.

7.2 Examination of Particle Size and its Potential

At room temperature, unmodified, RGD modified, R8 modified, RGD and R8 co-modified liposomes are taken and diluted 20 times, and injected into a sample cell. The average particle size and its distribution are measured with a laser particle size analyzer. The results show that the average particle size of the unmodified liposome is 153.4 nm, and the polydispersity coefficient (PDI) is 0.156, less than 0.3; the average particle size of the RGD modified liposome is 156.7 nm, and the PDI is 0.164, less than 0.3; the average particle size of the R8 modified liposome is 154.3 nm, and the PDI is 0.178, less than 0.3; and the average particle size of the RGD and R8 co-modified liposome is 155.2 nm, and the PDI is 0.102, less than 0.3; it is clear that the particle size distribution of each liposome is more concentrated.

8. Examination of Tumor Ball Penetratrion Ability of Co-Modified Liposome

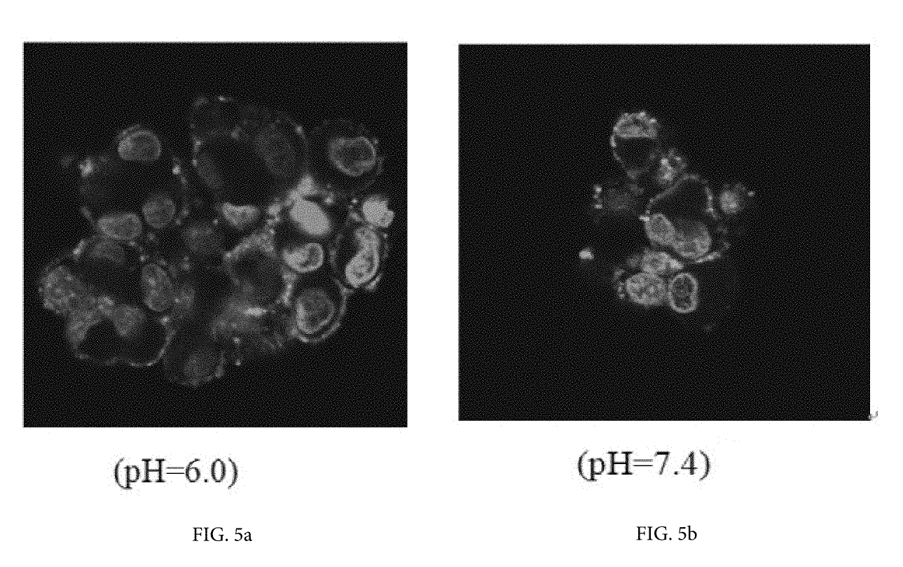

Since the tumor site tissues in vivo are multi-layer cells, in order to simulate its micro-environment in vivo, the present invention examines the ability of the co-modified liposome to penetrate the tumor balls under different pH conditions. 20 μ·mL⁻¹ of B27 serum-free medium, 20 ng·mL⁻¹ of EGF and bFGF (commercially available) are added to the culture medium, and the medium is half exchanged every 3 days. A mixture of the co-modified liposomes with a pH of 6.0 and pH of 7.4 and the culture medium are added to a 6-well plate containing tumor balls. After a co-incubation with tumor balls for 2 h, the tumor balls are pipetted, collected by means of centrifugation, washed with PBS 3 times, and added with Lyso-Tracker Red (commercially available) with a final mass concentration of 1 μg·mL⁻¹ for a co-incubation with the cells for 30 min The medium is then removed by means of centrifugation, washed 3 times with PBS, and fixed in 4% paraformaldehyde for 30 min The supernatant is removed by means of centrifugation, washed 3 times with PBS, and stained with 1 μg·mL⁻¹ of DAPI solution (commercially available) for 5 min The supernatant is removed by means of centrifugation, washed 3 times with PBS and the tumor balls are added to a polylysine-treated slide, and mounted in a 10% glycerol-PBS solution. FV 1000 laser confocal microscopy is used to observe the ability of co-modified liposome to penetrate the tumor balls. As shown in FIG. 5, the penetrating ability to tumor balls shows a significant pH dependence. Under an incubation condition where the pH is 6.0, the ability of the co-modified liposome to penetrate the tumor balls is significantly enhanced, compared with an incubation condition where the pH is 7.4.

9. Examination of the Uptake of Co-Modified Liposome by A549 Cells

Measurement of the Uptake Ratio by Flow Cytometry

The prepared FITC-labeled co-modified liposome with a pH of 7.4 is mixed quantitatively with 1640 medium (commercially available), so that the final dilution factors are 64, 96 and 128. The A549 cells are inoculated in a 6-well plate, and when the cells are fused to 80%, the former medium is pipetted, added with a mixture of unmodified, RGD modified, R8 modified, co-modified liposome and the culture medium. The culture medium is pipetted at 37° C. in a 5% CO₂ incubator after incubation for 2 h, washed 3 times with PBS, trypsinized to collect cells and washed 3 times with PBS. The supernatant is removed by centrifugation and finally added with 0.5 mL of PBS to re-suspend the cells. The uptake intensity of the co-modified liposomes at different concentrations are measured by BD flow cytometry. As shown in Table 10, the results show that given a co-modified liposome, the fluorescence intensity of the cell uptake is significantly higher than that of the unmodified and RGD or R8 modified liposome.

TABLE 10
Cell uptake of RGD and R8 co-modified ergosterol
and cisplatin active drug-loading liposome

| | | Uptake ratio |
|---|---|---|
| Groups | Dilution factor | (%) |
| Unmodified | 64 | 76.7% |
| liposome | 96 | 66.7% |
| | 128 | 47.1% |
| RGD modified | 64 | 81.4% |
| liposome | 96 | 60.1% |
| | 128 | 59.2% |
| R8 modified | 64 | 88.9% |
| liposome | 96 | 85.7% |
| | 128 | 73.0% |
| RGD and R8 | 64 | 98.9% |
| modified liposome | 96 | 96.2% |
| | 128 | 94.4% |

10. Examination of the Uptake Mechanism of Co-Modified Liposome

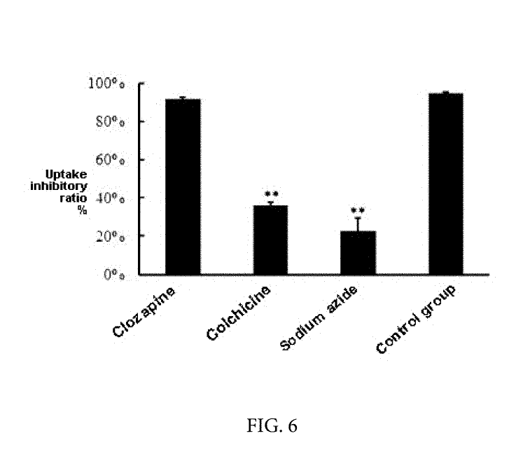

The A549 is inoculated into a 6-well plate and cultured at 37° C. in 5% CO₂ until the cells are fused to about 80%, added with 500 μg·mL⁻¹ of cell uptake inhibitors of clozapine, colchicine and sodium azide, respectively, wherein clozapine is a caveolin-mediated inhibitor through an endocytosis pathway, colchicine is an inhibitor through a macropinocytosis pathway, and sodium azide is an energy-dependent inhibitor through an endocytic pathway. The mixture of RGD and R8 co-modified liposome and the culture medium is then added to a 6-well plate after co-incubation with the cells for 30 min The medium is pipetted after 2 h, washed 3 times with PBS for a trypsin digestion and the cells are resuspended in 0.5 mL PBS after centrifugation. The degree of uptake of the co-modified liposome by the A549 cells after the addition of different inhibitors is examined by flow cytometry to examine the uptake and entry mechanisms of the co-modified liposome. The results in FIG. 6 show that the co-modified liposome can enter the cell through a variety of pathways, mainly an energy-dependent (azide) endocytic pathway and a macropinocytosis pathway (colchicine).

11. Influence of Inhibition of Co-Modified Liposome on A549 Cell Proliferation

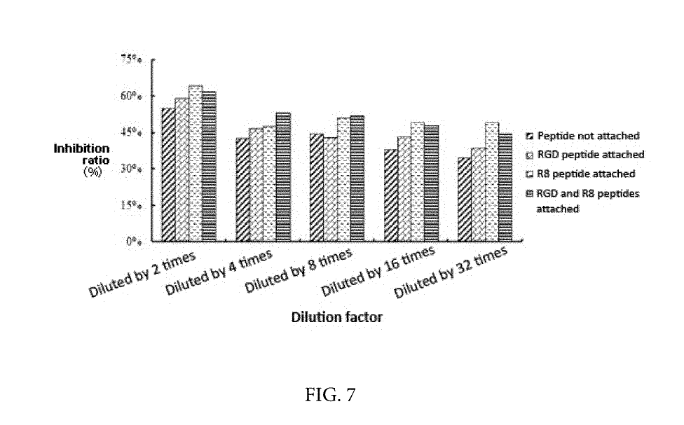

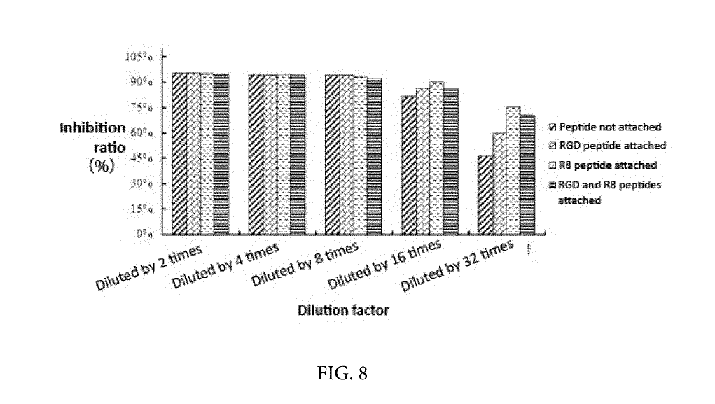

The A549 cells in a logarithmic growth phase are taken, with the number adjusted to 1×10⁵·mL⁻¹ after a trypsin digestion. 100 μL of the A549 cells are added in per well and inoculated in a 96-well culture plate, incubated at 37° C. in a 5% CO₂ incubator, and added with drugs for treatment when the cells are fused to 80%. The co-modified liposome with different dilution factors is added, while a normal control group is set with 5 complex holes for each concentration, respectively. An MTT assay is performed at 2 h and 24 h after the addition of drugs. An MTT (5 mg·mL⁻¹) solution (20 μL) is added to each well and incubated at 37° C. in a 5% CO₂ incubator for 4 h. The absorbency (OD) value in each well is measured at 492 nm with a microplate reader. When the drug acts for 2 h, the tumor cell proliferation inhibition ratios of the R8 modified and RGD and R8 co-modified liposomes are higher than those of the unmodified and RGD modified liposomes at a drug concentration diluted 2, 4, 8, 16 and 32 times (FIG. 7). When the action time is extended to 24 h, the inhibition ratios of the two groups have almost no difference when the dilution multiples are 2, 4 and 8 times, and they are all above 90%. When the dilution factors are 16 and 32, the inhibition conditions of each group are the same as those at 2 h (FIG. 8).

The results show that the penetrating peptide R8 and RGD peptide can efficiently mediate co-modified liposomes into the cell to release ergosterol and cisplatin drugs, so that the co-modified liposome has a stronger targeting property, making ergosterol and cisplatin drugs play a better role in inhibiting tumors.

The above-described embodiment is only a preferred embodiment of the present invention, and does not impose any limitation on the present invention. Other variations and modifications may be made without departing from the technical solutions recited in the Claims.

What is claimed is:

1. An RGD peptide and penetrating peptide R8 co-modified ergosterol and cisplatin active drug-loading liposome, characterized in that it is prepared by means of incubation of an ergosterol and cisplatin active drug-loading liposome, RGD cyclic peptide and penetrating peptide R8 in a water bath. The ergosterol and cisplatin active drug-loading liposome is prepared from an ergosterol liposome and a cisplatin solution serving as raw materials, wherein the mass ratio of ergosterol and cisplatin is controlled at 1:1-4:1.

2. The RGD peptide and penetrating peptide R8 co-modified ergosterol and cisplatin active drug-loading liposome as claimed in claim 1, characterized in that the ergosterol liposome is made of ergosterol (8-15 wt %) and liposome (85-92%), wherein the liposome is composed of lecithin and cholesterol, with a molar ratio of lecithin to cholesterol of 3:1-6:1.

3. The RGD peptide and penetrating peptide R8 co-modified ergosterol and cisplatin active drug-loading liposome as claimed in claim 2, characterized in that the ergosterol liposome is made of ergosterol (10 wt %) and liposome (90%), wherein the liposome is composed of lecithin and cholesterol, with a molar ratio of lecithin to cholesterol of 5:1.

4. The RGD peptide and penetrating peptide R8 co-modified ergosterol and cisplatin active drug-loading liposome as claimed in claim 1, characterized in that the amounts of RGD cyclic peptide and penetrating peptide R8 are controlled to be in a molar ratio of RGD cyclic peptide:penetrating peptide R8:cholesterol of 0.07:0.07:1.

5. The RGD peptide and penetrating peptide R8 co-modified ergosterol and cisplatin active drug-loading liposome as claimed in claim 1, characterized in that the RGD cyclic peptide is specifically DSPE-PEG3400-c; the penetrating peptide R8 is specifically DSPE-PEG1000-R8.

6. The RGD peptide and penetrating peptide R8 co-modified ergosterol and cisplatin active drug-loading liposome as claimed in claim 1, characterized in that the RGD peptide and penetrating peptide R8 co-modified ergosterol and cisplatin active drug-loading liposome is prepared by means of incubation of an ergosterol and cisplatin active drug-loading liposome, RGD cyclic peptide and penetrating peptide R8 in a water bath at 55° C. for 1 h.

7. The RGD peptide and penetrating peptide R8 co-modified ergosterol and cisplatin active drug-loading liposome as claimed in claim 1, characterized in that the cisplatin solution has a concentration of 0.03-0.3 mg/mL.

8. The RGD peptide and penetrating peptide R8 co-modified ergosterol and cisplatin active drug-loading liposome as claimed in claim 7, characterized in that the cisplatin solution has a concentration of 0.15 mg/mL.

9. The RGD peptide and penetrating peptide R8 co-modified ergosterol and cisplatin active drug-loading liposome as claimed in claim 1, characterized in that a specific method of preparing the ergosterol and cisplatin active drug-loading liposome is as follows: (1) preparation of ergosterol liposome: lecithin, cholesterol and ergosterol are weighed, dissolved in chloroform, rotavaporized into a thin film, dried in a vacuum, hydrated with an ammonium chloride solution as a hydration solution and ultrasonically released; a probe is ultrasonically treated in an ice bath, filtered and extruded under a high pressure to generate an ergosterol liposome; (2) gradient formation of ammonium chloride: the ergosterol liposome is added to a dialysis bag with a molecular weight cut off of 8000-14000 Da; the dialysis bag is closed and dialyzed in distilled water for 2 h, and the distilled water is changed once to continue the dialysis for 2 h; (3) drug loading: cisplatin is mixed with water to make a cisplatin solution, and the dialyzed ergosterol liposome is added to the cisplatin solution for incubation, wherein the amount of ergosterol liposome added satisfies a mass ratio of ergosterol to cisplatin is 1:1 to 4:1, and the product is generated after the incubation at an incubation temperature of 40-60° C. and an incubation time of 5-40 min

10. The RGD peptide and penetrating peptide R8 co-modified ergosterol and cisplatin active drug-loading liposome as claimed in claim 9, characterized in that the parameters of the ultrasonic treatment of the probe in Step (1) are as follows: ultrasonic time: 20 min, ultrasonic: 2s, stop: ls, ultrasonic power: 900W and pressure for the high-pressure extrusion: 400-500 psi.

11. The RGD peptide and penetrating peptide R8 co-modified ergosterol and cisplatin active drug-loading liposome as claimed in claim 9, characterized in that the amount of the ergosterol liposome added in Step (3) is calculated to satisfy that the mass ratio of ergosterol to cisplatin is 2.5:1, the incubation temperature is 50° C. and the incubation time is 10 min

12. The RGD peptide and penetrating peptide R8 co-modified ergosterol and cisplatin active drug-loading liposome as claimed in claim 9, characterized in that the concentration of the ammonium chloride solution in Step (1) is 0.1-1.5 mmol·L⁻¹.
